(19) United States
(12) Patent Application Publication
(10) Pub. No.: US 2008/0004462 A1
(43) Pub. Date: Jan. 3, 2008

(54) Catalyst for the Preparation of Fumaronitrile and/or Maleonitrile

(76) Inventors: Alexander V. Peters, Aachen (DE); Peter A.C. Schevelier, Hulsberg (DE)

Correspondence Address: NIXON & VANDERHYE, PC, 901 NORTH GLEBE ROAD, 11TH FLOOR, ARLINGTON, VA, 22203, US

(21) Appl. No.: 11/667,960

(22) PCT Filed: Nov. 21, 2005
(86) PCT No.: PCT/EP05/12496

§ 371 Date: Jul. 17, 2007

(30) Foreign Application Priority Data
Nov. 22, 2004 (EP) 04078186.6

(51) Int. Cl. C07C 253/24; B01J 21/08; B01J 23/30

(52) U.S. Cl. 558/319; 502/242

(57) ABSTRACT

The invention relates to a catalyst comprising a titanium dioxide carrier and a mixture of metal oxides comprising at least one oxide of a metal selected from the group consisting of vanadium and tungsten and silicon oxide, comprised in such an amount that silicon (Si) is present in the catalyst in an amount of at least 1.0 wt %, relative to the weight of the catalyst. The invention also relates to a process for the preparation of fumaronitrile and/or maleonitrile by ammoxidation of C₄-straight chain hydrocarbons in the presence of a catalyst according to the invention.

The invention relates to an ammoxidation catalyst comprising a carrier and a mixture of metal oxides comprising at least one oxide of a metal selected from the group consisting of vanadium and tungsten and at least one oxide of a further element. The invention also relates to a process for the preparation of fumaronitrile and/or maleonitrile by ammoxidation of C₄-straight chain hydrocarbons in the presence of said catalyst

Such a catalyst is known from U.S. Pat. No. 4,436,671. The known catalyst consists essentially of the following active components:
(A) at least one oxide of vanadium (V) and tungsten (W), and
(B) (1) at least one oxide of antimony (Sb), phosphorus (P) and boron (B), and/or
   (2) at least one oxide of chromium (Cr), nickel (Ni), aluminum (Al) and silicon (Si).

As carrier or support for the known catalyst alumina, titanium oxide or titanium phosphate, amongst others, can be used. The known catalyst can be prepared from various compounds of the respective elements using methods known per se. The known catalyst has been prepared by wet-moulding into a granule shape with a granule diameter of 2 mm and a length of 5 mm. The known catalyst is used in a fixed bed process for the preparation of fumaronitrile and/or maleonitrile by ammoxidation of C₄-straight chain hydrocarbons. As the substrate for the known process C₄-straight chain hydrocarbons were used, particularly butane, butene, butadiene or their mixtures.

The use of the known catalyst in the known process results in variable yields of fumaronitrile plus maleonitrile, depending on the specific catalyst composition. Generally, the yield is between 15 and 42%, and on average 26% for the larger number of experiments. Only in a few experiments a higher yield is obtained. Typically, the catalysts used in these examples contain oxides of tungsten (W) and vanadium (V) (with a total of the active species of these metal elements of about 2.3 wt. %) next to oxides of P (about 6.7 wt. %), Cr and/or Ni (with a total of about 0.6 wt. %) Next to these elements the catalysts comprise traces of other elements originating from the W-source used for the preparation of the catalyst. Catalysts comprising the same or similar elements but giving much lower yields included catalysts with a W content of about 4 wt. %, while no V is present, a P content of about 3.8 wt. % in combination with a Sb content of about 3 wt. %.

A disadvantage of the known catalyst is that it is very difficult to reproduce the performance of the catalyst and to obtain high yields in ammoxidation reactions; for example when the catalyst has a different form than granules, such as a powder, and the catalyst is used in a process for the preparation of fumaronitrile and/or maleonitrile by ammoxidation of C₄-straight chain hydrocarbons, the yield of fumaronitrile plus maleonitrile is much lower than mentioned in U.S. Pat. No. 4,436,671 and too low for use in industrial processes.

The aim of the invention is therefore to provide a catalyst, which gives a better yield than the known catalyst when used in a powder form.

This aim has been achieved with the catalyst according to the invention, wherein the carrier is titanium dioxide and the catalyst comprises silicon oxide in such an amount that silicon (Si) is present in the catalyst in an amount of at least 1.0 wt %, relative to the weight of the catalyst.

The effect of the catalyst according to the invention wherein the carrier is titanium dioxide and the silicon (Si) is present in an amount of at least 1.0 wt %, relative to the weight of the catalyst, is that when the catalyst is used in powder form the yield of fumaronitrile plus maleonitrile is higher than with the known catalyst prepared in powder form. Improved results are obtained over a wide range of compositions of the gas feed supply.

By contrast, when a catalyst in powder form is used with a regular amount of tungsten and a high amount of P, as high as in one of the better catalysts in U.S. Pat. No. 4,436,671, the yield of fumaronitrile plus maleonitrile is much lower than with the catalyst according to the invention and also much lower than the results reported for catalysts with granule shape and similar compositions reported in U.S. Pat. No. 4,436,671.

A powder is herein understood to be a material consisting of particles with a small particle size. Typically such a material has a particle size distribution with the majority of the particles have a particle size of for example, of at most 2 mm. Suitably, the catalyst according to the invention has the form of a particle shaped powder, with a median particle size (d₅₀) of at most 2 mm, meaning that 50% or more of the weight of the particles has a particle size of at most 2 mm. The median particle size can be determined with the use of sieves. Suitable test methods for determining the median particle size are, for example, test methods according to ASTM4570-86 and ASTMD5644-96.

Preferably, median particle size of the catalyst according to the invention is at most 1 mm, and said median particle size may be very well be as low as 0.5 mm or lower. In a preferred embodiment, the median particle size is 0.05-0.2 mm.

Preferably, the amount of silicon in the catalyst according to the invention is at least 1.5 wt. %, more preferably at least 2.0 wt % and most preferably at least 4.0 wt %. A higher minimum amount of silicon in the catalyst according to the invention results in a higher yield of fumaronitrile plus maleonitrile in the described ammoxidation process. The amount of silicon may be as high as 10 wt. % or higher, but amounts well above 10 wt. % only lead to an incremental increase of the yield of fumaronitrile plus maleonitrile.

In the catalyst according to the invention, the carrier is titanium dioxide. Preferably, the titanium dioxide consists of particles with a median particle size (d₅₀) of at most 2 mm, more preferably at most 1 mm, more preferably at most 0.5 mm. In a preferred embodiment, the median particle size is 0.05-0.2 mm.

The catalyst according to the invention comprises at least one oxide of tungsten and vanadium. Preferably, the inventive catalyst comprises a combination of at least one tungsten oxide and at least one vanadium oxide. Also preferably, the inventive catalyst comprises tungsten and/or vanadium in a total amount of 0.1-10 wt. %, relative to the weight of the catalyst. Suitably, the total amount of tungsten and/or vanadium is 1-5 wt. %, more preferably 2-3 wt. %.

The inventive catalyst may optionally comprise further active components. Preferably, the catalyst further comprises oxide compounds of P and/or Cr. These oxide compounds may have any suitable form, for example, an acid or a metal oxide. A suitable acid is, for example, phosphoric acid. A suitable oxide is, for example, chromium trioxide. Preferably, the inventive catalyst comprises a combination of at least one phosphorus oxide and at least one chromium oxide. Also preferably, the inventive catalyst comprises phosphorus and/or chromium in a total amount of 0.1-15 wt. %, relative to the weight of the catalyst. Suitably, the total amount of tungsten and/or vanadium is 1-12 wt. %, more preferably 5-10 wt. %.

Also preferably, the catalyst further comprises an oxide of an element chosen from the group consisting of Cu, Fe, Ni, Na, K and mixtures thereof.

For the preparation of the catalyst according to the invention any method that is suitable for preparing metal oxide based catalysts may be used. In these methods the silica may added as such or may be formed in-situ. When the silica is added as such it may be added, for example, in the form of a silica sol (e.g. Ludox® silica sol, available from Grace), as a solution of a silicate (e.g. sodium silicate), as a powder of various types of silica gel or precipitated silica, or as a fine powder of pyrogenic silica (e.g. so called “aerosil”), produced by flame hydrolysis of e.g. SiCl4. The silica may be added to a slurry of a catalyst containing the oxides of the other metal components, or to a slurry containing a mixture of oxides of the other metal components. Alternatively, the catalyst containing the oxides of the other metal components or the mixture of oxides of the other metal components may also be added to a slurry containing finely dispersed silica, or to a slurry containing silica particles on the carrier material. Silica can also be formed in situ by hydrolysis of organic silicon-containing compounds, e.g. tetra-ethylorthosilicate (TEOS, Si(OC2H5)4). The silica may be formed in-situ by adding a organic silicon-containing compound to a slurry of a catalyst containing the oxides of the other metal components in water or in a water-containing liquid mixture, or to a slurry containing a mixture of oxides of the other metal components in water or a water-containing liquid mixture. After a combined slurry, containing both the silica and the catalyst containing the oxides of the other metal components or both the silica and the mixture of oxides of the other metal components, is formed, the silica and the catalyst containing the oxides of the other metal components or the silica and the mixture of oxides of the other metal components may be co-precipitated. Co-precipitation may be carried, for example, by spray drying, thereby forming a dry powder. Co-precipitation may also be carried out by wet molding into granules, as described in U.S. Pat. No. 4,436,671, followed by grinding to form a powder.

The catalyst can also be prepared by dissolving a silicium containing compound, a tungsten and/or vanadium containing compound, and optionally compounds of further active elements, which can all be converted into oxides by chemical reaction or heating, in an appropriate solvent such as water, alcohols, acids, and alkalis, if necessary, and then allowing them to be impregnated or deposited on a carrier material, followed by calcination at a temperature ranging from 300° C. to 800° C.

Preferably, the silica is added in the form of a silica sol. This has the advantage that a high concentration of very well dispersed silica can be added in a simple way to a base catalyst comprising the titanium dioxide and the other metal oxides.

The invention also relates to a process for the preparation of fumaronitrile and/or maleonitrile by ammoxidation of C₄-straight chain hydrocarbons in the presence of a catalyst comprising an oxide of silicon (Si) and at least one oxide of vanadium and tungsten. The catalyst used in the process according to the invention is the catalyst according to the invention or any of the preferred embodiments thereof.

The advantage of the process according to the invention, or the preferred embodiments thereof have the advantages as described above for the inventive catalysts.

The process may be carried out as a batch process or as a continuous process, and as a fixed bed process or fluidized bed process.

The invention is further illustrated with the following Examples and Comparative Experiments.

Materials

Titanium dioxide: Degussa P 25
Silica sol: Ludox® silica sol, sol of silica particles in water, diluted with water to a solids content of 25 wt. %, relative to the total weight of the sol.
Metal compounds: laboratory grades were used.
Silicium carbide: Industrial grade with an average particle diameter of 0.29 mm
   Preparation of the Catalysts
   Catalyst I for Example I

To 1600 grams of distilled water were added 47.61 grams of silicotungstic acid, 15,7 grams of vanadium pentoxide and 196 grams of oxalic acid. The mixture was heated to 80° C. under continuous stirring and kept at 80° C. to obtain a homogeneous solution (solution 1). Another solution was prepared by dissolving 17.3 grams of chromium trioxide and 451.8 grams of 85% phosphoric acid in 3000 grams of distilled water (Solution 2). To this solution was added carefully 1050 grams of fine titanium dioxide powder, resulting in a titanium dioxide containing dispersion. The dispersion was homogenized by stirring and subsequently solution 1 was added while stirring. Finally 640 grams of silica sol, with 25 wt. % silica particles, was added. Addition was performed slowly under vigorous stirring. Then, the resulting dispersion was spray dried using a small scale R&D type spray dryer. The spray-dried powder was calcined at 500° C. for 4 hours. The resulting powder had a median particle size of 30-40 μm.

Catalyst A for Comparative Experiment A

Catalyst A was prepared by using the method of Catalyst I, except that the addition of the silica sol was left out. The composition of the Catalyst I and Catalyst A, as determined with XRF for the main metallic elements, is given in Table 1.

TABLE 1
composition of catalysts
(in wt % relative to the weight of the catalyst).

| Element | Catalyst A | Catalyst I |
|---|---|---|
| Ti | 42 | 37 |
| W | 2 | 1.7 |
| P | 7.5 | 7 |
| Cr | 0.5 | 0.5 |
| Trace elements (total) | 0.05 | 0.25 |
| Si | — | 4.5 |

Description of Ammoxidation Test
Products Used

Liquefied 1,3-butadiene, stabilized with p-TBC, and liquefied ammonia in cylinders were used as sources of the respective gases. The purity of 1,3-butadiene was 99.7% v/v; the quality of the ammonia used was UHP (ultra high purity) 99.998% v/v.

Purified air and high purity nitrogen were taken from a general laboratory supply source.

The fumaronitrile used for calibration was from Fluka (purum>99% GC), the maleonitrile used for the same purpose was specially synthesized and 98% pure after recrystallisation. Also, a gaseous mixture of 1% v/v of 1,3-butadiene and 99% v/v high purity nitrogen was used for calibration purposes.

Silicon carbide, having an average particle diameter of 0.29 mm, was used as support for the catalyst bed. Silicium carbide is inert in respect of ammoxidation reactions.

Equipment

The ammoxidation was carried out in a flow-type fixed-catalyst bed quartz reactor with 15 mm inner diameter.

The reactor was heated by means of a thermoregulated electrical heating oven, the temperature being measured in the catalyst bed. The gaseous feed consisting of 1,3-butadiene, ammonia, and a mixture of air and nitrogen was supplied to the reactor by means of mass flow controllers.

The off-gas from the top of the reactor was divided into two streams. The main stream was treated in a scrubber with alkali in order to trap the hydrogen cyanide produced and the final oxygen concentration in the product mixture was measured in this stream with an oxygen meter, type PMA 30, M&C Instruments, Bleiswijk, The Netherlands. The second stream was sent to a gas chromatograph to analyze the amount of fumaronitrile and maleonitrile formed using a CPSil5CB column with FID detector and to analyze unrelated butadiene using a CPSil5CB and Porabond Q column with a TCD detector.

The mass flow controllers, the oxygen meter and the GC were calibrated before starting each series of experiments.

Test Conditions and Procedure

Between 0.5 and 3.0 g catalyst per charge were tested. A weighed amount of catalyst was diluted to 10 ml with silicon carbide and packed into the reactor, which was then filled-up completely with silicon carbide, having an average particle diameter of 0.29 mm.

The catalysts were tested at atmospheric pressure and 560° C. For this purpose, a flow of air was applied to the reactor and temperature was slowly increased to 560° C. The oxygen content in the gas mixture was controlled by supplement of an additional nitrogen stream. Then ammonia and butadiene were added to the gas stream in this order until a total gas flow of 30 Nl/h was reached, and kept constant at this level, resulting in SV=3000 h⁻¹ on the basis of a diluted catalyst bed volume of 10 ml. The mole ratio of 1,3-butadiene: ammonia:air:nitrogen was varied between the following limits: 0.33 to 0.50 (1,3-butadiene): 1.17 to 5.00 (ammonia): 20.0 to 97.0 (air): 0 to 77.0 (nitrogen). The resulting catalyst load ranged from 2.2 to 6.3 mmole butadiene/gcat.h.

Consecutive experiments were carried out on a one-experiment-per-day basis. In each one-day experiment, a certain mole ratio of feed gases was chosen and the off gas was analyzed by GC until composition became constant. A different mole ratio was then applied, the off gas was analyzed again, and so on until the end of the series of experiments for a single catalyst charge. Finally, conversion of butadiene, selectivity and yield of fumaronitrile and maleonitrile were calculated based on known feed rates and measured unreacted butadiene and reaction products measured.

Product peaks in chromatograms were identified by retention times and by means of observed peak area increase upon standard addition.

Ammoxidation Experiments.

The gas feed compositions used in the experiments, as well as the oxygen content measured for the off gases in the individual experiments have been summarized in Table 2. The conversion (X), selectivity (S) and yield (Y) for the butadiene conversion and succinontril formation measured under different conditions have been reported in Table 3.

### EXAMPLE I

In example I catalyst I was used. The results measured under the various conditions have been reported in Table 2, column 6 and Table 3, columns 2-4 (Exp 23 Analysis 1-4).

### Comparative Experiment A

For Comparative Experiment A Example I was repeated except that the catalyst I used in Example I was replaced by catalyst A. The results have been collected in Table 2, column 7 and Table 3, columns 5-7 (Exp 22, Analysis 1-4).

TABLE 2
Gas feed and gas feed composition

| | | | | | Example I | Comparative |
|---|---|---|---|---|---|---|
| Gas feed | Qin | Bu | NH₃ | Air | O2 measured | Example A; O2 |
| composition* | (N1/h) | (vol %) | (vol %) | (vol %) | (vol %) | measured (vol %) |
| 1 | 30 | 0.50 | 2.50 | 29.67 | 4.4 | 4.7 |
| 2 | 30 | 0.50 | 4.00 | 53.33 | 9.4 | 9.7 |
| 3 | 30 | 0.50 | 5.00 | 94.49 | 18.3 | 18.8 |
| 4 | 30 | 0.50 | 2.50 | 29.67 | 4.4 | 4.6 |

*Remainder of the composition of gas feed was the nitrogen gas flow, making up for the rest of the 100% in total.

TABLE 3
Conversion of butadiene, selectivity and yield of succinonitrile

| | Results | | | | | | |
|---|---|---|---|---|---|---|---|
| | Exam- | | | Results | | | Calculated |
| Gas feed | ple I | S | Y | CE A | S | Y | Delta |
| composition | X (%) | (%) | (%) | X (%) | (%) | (%) | Y (%) |
| 1 | 95 | 38 | 36 | 88 | 35 | 31 | 5 |
| 2 | 94 | 40 | 37 | 86 | 36 | 31 | 6 |
| 3 | 94 | 53 | 50 | 87 | 52 | 46 | 4 |
| 4 | 95 | 61 | 58 | 90 | 56 | 53 | 5 |

Comparison of the results for Example I and Comparative Experiment A in Table 3 shows that the catalyst according to the invention, i.e. catalyst I of Example I, gives better conversion and selectivity and better overall yields for all conditions tested than catalyst A in Comparative Experiment A. With the catalyst according to the invention the absolute yield is increased with about 5%, and on a relative scale even 6 to 9%, which is quite substantial when applied to an industrial process.

1. Catalyst comprising a carrier and a mixture of metal oxides comprising at least one oxide of a metal selected from the group consisting of vanadium and tungsten and at least one oxide of a further element, characterized in that the carrier is titanium dioxide and the at least one oxide of a further element is silicon oxide, comprised in such an amount that silicon (Si) is present in the catalyst in an amount of 1.0-10 wt % relative to the weight of the catalyst.

2. Catalyst according to claim 1, wherein the catalyst has the form of a particle shaped powder, with a median particle size (d₅₀) of at most 2 mm.

3. Catalyst according to claim 1, wherein the amount of silicon is at least 1.5 wt %, preferably at least 2 wt. %, relative to the weight of the catalyst.

4. Catalyst according to claim 1, wherein the catalyst further comprises P and/or Cr.

5. Catalyst according to claim 1, wherein the catalyst further comprises an oxide of an element chosen from the group consisting of Cu, Fe, Ni, Na, K and mixtures thereof.

6. Use of the catalyst according to claim 1 for the preparation of fumaronitrile and/or maleonitrile.

7. Process for the preparation of fumaronitrile and/or maleonitrile by ammoxidation of C₄-straight chain hydrocarbons in the presence of an ammoxidation catalyst comprising a carrier and at least one oxide of a metal selected from the group consisting of vanadium and tungsten, and at least one oxide of a further element characterized in that the ammoxidation catalyst is a catalyst according to claim 1.
